(19) United States
(12) Patent Application Publication
(10) Pub. No.: US 2018/0235977 A1
(43) Pub. Date: Aug. 23, 2018

(54) Treatment for Microbial borne illness

(71) Applicant: Michael Earl Drabinsky, Walnut Creek, CA (US)

(72) Inventor: Michael Earl Drabinsky, Walnut Creek, CA (US)

(21) Appl. No.: 15/436,854

(22) Filed: Feb. 19, 2017

(51) Int. Cl. A61K 31/5383; A61K 31/435; A61K 31/4164; A61K 9/00; A61K 45/06

(52) U.S. Cl. CPC A61K 31/5383; A61K 31/435; A61K 45/06; A61K 9/0053; A61K 9/0014; A61K 31/4164

(57) ABSTRACT

A method and a pharmaceutical composition for treating Bartonella, Eubacteria or Archaea borne illnesses administered to a patient in need of such treatment at least one of tinidazole dissolving bacterial biofilm in combination with of fluoroquinolone and a rifamycin class drug killing a bacterial infection in conjuction with palliative remedies for treating herx.

### CROSS REFERENCE TO RELATED APPLICATIONS

This application claims the benefit of and incorporates by reference continuance in part U.S. provisional patent application Ser. No. 62/296,681 titled TINIDAZOLE DRUG COMBINATIONS FOR FIGHTING PATHOGENS filed Feb. 21, 2016.

### BACKGROUND OF THE INVENTION

### Field of the Invention

This invention relates to a combination of two or more short and long acting anti-pathogenic compounds, including tinidazole, which dissolve the protective biofilm barriers of microbes found within the blood vessel walls or any nearby tissue to treat Bartonella and Bartonella-like organisms (BLO) and other bacterial infections such as Eubacteria or Archaea

Bartonella usually manifests clinically as any kind of nerve problem in any part of the body.

For example, a Bartonella infection in the brain may cause seizures and epilepsy. In a foot, Bartonella may cause neuropathy or diabetic nerve pain. Bartonella can also cause psychiatric changes from mood swings to schizophrenia and autism. In severe cases Bartonella is usually associated with Borrelia or other infections.

Microorganisms generally live attached to surfaces in many natural, industrial, and medical environments, encapsulated by extracellular substances including biopolymers and macromolecules. The resulting layer of slime-encapsulated microorganism is termed a biofilm. A biofilm is any group of microorganisms in which cells stick to each other on a surface. These adherent cells are frequently embedded within a self-produced matrix of extracellular polymeric substance (EPS), Biofilms are the predominant mode of growth of bacteria in the natural environment, and bacteria growing in biofilms exhibit distinct physiological properties. Compared to their planktonically grown counterparts, the bacteria in a biofilm are more resistant to antibiotics, UV irradiation, detergents and the host immune response (Gristina et al. 1988. Journal of the American Medical Association, 259: 870-874; Stewart. 1994. Antimicrobial Agents & Chemotherapy, 38(5): 1052-1058; Costerton et al. 1995. Annu. Rev. Microbiol., 49: 71 1-745; Maira Litran et al. 2000. Journal of Applied Microbiology, 88: 243-247). A biofilm may include one or more microorganisms, including gram-positive and gram-negative bacteria, algae, protozoa, and/or yeast or filamentous fungi and viruses and/or bacteriophage. Examples of problematic biofilms are dental plaque, infections on medical implants, but also the initial fouling on ship hulls (Satuito et al. 1997. Hydrobiologia, 358: 275-280). Biofilms are attributed to the pathogenesis of many infections in humans. New strategies are required to disperse existing biofilm.

Many types of microbes grow naturally in a biofilm context, such as bacteria, fungi, algae etc.

It is known in the art that biofilms can have, as a component, DNA (termed extracellular DNA or eDNA) although its function there remains unknown. Certain groups have sought to employ nuclease enzymes to disrupt biofilms.

Tinidazole is the best molecule currently known for dissolving biofilms. Metronidazole (Flagyl) is in the same drug class as tinidazole and can work in a similar fashion, however Flagyl is not as effective as Tindamax for dissolving biofilms and Flagyl has more adverse side effects.

Other compounds such as nattokinase and serrapeptase can dissolve biofilms, however they are not very effective in a patient treatment setting even though nattokinase and serrapeptase are less expensive than Tindamax.

Tindamax eliminates biofilm from the body because atmospheric biofilm disruption does not reduce the total volume of biofilm in one's body, but rather, it breaks apart larger chunks of biofilm into smaller chunks.

Anti-microbials are used concurrently with biofilm disruption techniques to kill the microbes that are released when biofilm is broken apart or destroyed because when biofilm is dissolved, pathogenic organisms are usually released into the body or bloodstream. Otherwise, if biofilm is broken up too rapidly, several things can happen. First, pathogens may be released into the bloodstream so quickly that sepsis ensues which can be harmful (life-threatening) to the patient. Second, rapid physical ‘chopping’ of large biofilm chunks can break them apart into smaller chunks which can then block blood vessels causing a whole bunch of problems such as heart attack, stroke, TIA, seizure, organ damage/failure due to lack of blood flow, etc.

In order to prevent further harm to a patient, methods of getting rid of biofilm should gradually be ramped up so there is a slow, safe, controlled release of biofilm chunks. Also, there is usually a high density of individual pathogens within a single biofilm chunk that get released when conducting a treatment to get rid of biofilm.

Tinidazole is only good at destroying biofilm and it is very limited in killing actual organisms.

Although it may be considered obvious on the broader level to combine tinidazole with antibiotics for treating Bartonella or similar infections, in particular, the removal of biofilms currently poses significant problems since the bacteria present in the biofilm are highly resistant to many antimicrobial compounds. Furthermore, prior art methods for biofilm disruption involve compositions active against mainly only gram negative proteobacteria, and show very specific activity against a limited number of strains. This significantly limits their utility. Thus, there remains a need for new biofilm disruption and treatment involving compositions with improved properties.

Although it may be considered obvious on the broader level to combine tinidazole with antibiotics for treating Bartonella or similar infections, in particular, the removal of biofilms currently poses significant problems since the bacteria present in the biofilm are highly resistant to many antimicrobial compounds. Furthermore, prior art methods for biofilm disruption involve compositions active against mainly only gram negative proteobacteria, and show very specific activity against a limited number of strains. This significantly limits their utility. Thus, there remains a need for new biofilm disruption and treatment involving compositions with improved properties.

### DESCRIPTION OF TIE PREFERRED EMBODIMENTS

As with the other patents, a combination of drugs is required to eradicate Bartonella infection. At least one biofilm disruption drug (tinidazole) and one bactericidal drug (Levaquin) are necessary. Usually a third antibiotic (rifabutin or rifampicin) is also necessary.

The current invention uses the brand name Tindamax (tinidazole), marketed by Mission Pharmacal, or Fasigyn and Simplotan marketed by Pfizer, which attacks the biofilm that the bacteria use to protect themselves, rendering most drug treatments useless.

The chemical name for Tinidazole is C₈H₁₃N₃O₄S.

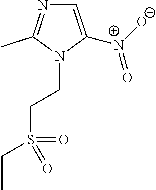 [SMILES: CCS(=O)(=O)CCn1c([N+](=O)[O-])cnc1C]

In prior art systems, the biofilm stops the drugs from reaching the bacteria, and this prevents their treatment regardless of which drugs are used. Tinidazole should be used to dissolve biofilms. Other nitroimidazole class drugs may be used instead of tinidazole to dissolve biofilm.

The chemical name for nitroimidazole is C H₃N₃O₂

This invention uses three (or more) drug treatments for Bartonella infections. The current invention uses the brand name Tindamax (tinidazole), marketed by Mission Pharmacal, or Fasigyn and Simplotan marketed by Pfizer, which attacks the biofilm that microorganisms use to protect themselves, rendering most drug treatments useless. In prior art systems, the biofilm stops the drugs from reaching microorganisms, and this prevents their treatment regardless of which drugs are used.

The best treatment options are as follows:
1. A biofilm disruption drug such as tinidazole or metronidazole;
2. A late generation fluoroquinolone such as levofloxacin. The chemical name for levofloxacin is C₁₈H₂₀FN₃O₄. Levofloxacin works better against Bartonella than does ciprofloxacin. There are many fluoroquinolones that may work against Bartonella and Borrelia; and
3. A rifamycin class drug such as rifabutin or rifampicin is also a good Bartonella killer. The chemical name for Rifabutin is C₄₆H₆₂N₄O₁₁ and it works better and has fewer side effects and drug interactions than rifampicin does.

An example of a good Bartonella treatment regimen would be to take tinidazole, levofloxacin, and rifabutin simultaneously as combination therapy for Bartonella.

Each drug has its own half-life and some drugs may need to be dosed more or less frequently than others based on half-life. Also some drugs, especially rifampicin, may affect the blood concentration levels of other drugs. Therefore, dosing of each drug may need to be increased or decreased.

As with the other drug combinations for treatment of infections there may be moderate to severe Jarish-Herxheimer (herx) reactions. Palliative remedies such as pain medications, glutathione, tricyclic antidepressants, SSRI's, benzodiazepines, etc. are usually required to make the herx tolerable. For neuropathy of the skin, the best and most cost effective remedy can be Dead Sea salt or mud products. For example, AHAVA is the only company licensed to mine from the Dead Sea and sells lotions, creams, etc. that can offer near 100% relief of diabetic nerve pain or neuropathy.

Whilst the prior art recognized that antibiotics are used to treat Bartonella and other biofilm producing pathogens and amoebas. However, none of the prior art used a Tinidazole drug combination for the treatment of Bartonella et al to break down biofilms that pathogens protect themselves with, alongside antibiotics that can destroy the pathogens.

Thus, the invention provides a pharmaceutical composition for disrupting a biofilm or preventing biofilm formation comprising a Tinidazole drug combination.

In any of the pharmaceutical compositions described herein the composition can be formulated for oral administration.

In any of the pharmaceutical compositions described herein the composition can be formulated as a liquid, lotion, cream, spray, gel, ointment, or powder, and the like.

Any of the pharmaceutical compositions describe herein can be formulated for use in the treatment of a wide range of medical indications.

Where the composition is a pharmaceutical composition, said composition can be for administration to an animal patient. The animal patient can be a mammalian patient. The mammalian patient can be a human.

The invention also provides a method of disrupting a biofilm on a patient comprising contacting a biofilm on a patient with any of the pharmaceutical compositions described herein.

The patient can be an animal patient. The patient can be a mammalian patient. The patient can be a human.

In any of these methods the biofilm can comprise gram-positive bacteria.

Examples of formulations include topical lotions, creams, soaps, wipes, and the like. They may be formulated into liposomes, to reduce toxicity or increase bioavailability. Other methods for delivery include oral methods that entail encapsulation of the polypeptide or peptide in microspheres or proteinoids, aerosol delivery (e.g., to the lungs), or transdermal delivery (e.g., by iontophoresis or transdermal electroporation). Other routine methods of administration will be known to those skilled in the art.

Pharmaceutical formulations, containing any of the compositions described herein, suitable for oral administration may be provided in convenient unit forms including capsules, tablets, gels, pastes, ointments etc.

Preparations for parenteral administration of pharmaceutical formulations comprising compositions described herein include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, antioxidants, chelating agents, inert gases and the like also can be included.

For topical administration to the epidermis, any of the pharmaceutical compositions may be formulated as an ointment, cream, or lotion. Ointments and creams, may, for example, be formulated with an aqueous or oily base with the addition of suitable thickening and/or gelling agents. Lotions may be formulated with an aqueous or oily base and will in general also contain one or more emulsifying agents, stabilizing agents, suspending agents, thickening agents, or coloring agents.

While there have been described what are presently believed to be the preferred embodiments of the invention, those skilled in the art will realize that various changes and modifications of another molecule or drug of a similar family may be made to the invention without departing from the spirit of the invention, and it is intended to claim all such changes and modifications as fall within the scope of the invention.

1) A method for treating Bartonella borne illnesses comprising administering to a patient in need of such treatment at least one of tinidazole dissolving bacterial biofilm in combination with of fluoroquinolone and a rifamycin class drug killing a bacterial infection.

2) The method of claim 1 further comprising administering fluoroquinolone from a group consisting of levofloxacin and ciprofloxacin.

3) The method of claim 1 further comprising administering a rifamycin class drug. from a group consisting of rifabutin and rifampicin.

4) The method of claim 1 further comprising palliative remedies for treating herx.

5) The method of claim 4 wherein the palliative remedies further comprising from a group consisting of pain medications, glutathione, tricyclic antidepressants, SSRI's, and benzodiazepines.

6) The method of claim 1 further comprising treating Eubacteria or Archaea borne illnesses.

7) A pharmaceutical composition for treating Bartonella, Eubacteria or Archaea borne illnesses comprising administering to a patient in need of such treatment at least one of tinidazole dissolving bacterial biofilm in combination with of fluoroquinolone and a rifamycin class drug killing a bacterial infection.

8) The pharmaceutical composition of claim 7 further comprising other fluoroquinolone from a group consisting of levofloxacin and ciprofloxacin.

9) The pharmaceutical composition of claim 7 further comprising a rifamycin class drug from a group consisting of rifabutin and rifampicin.

10) The pharmaceutical composition of claim 7 further comprising palliative remedies for treating herx.

11) The pharmaceutical composition of claim 10 wherein the palliative remedies further comprising from a group consisting of pain medications, glutathione, tricyclic antidepressants, SSRI's, and benzodiazepines.

12) The pharmaceutical composition of claim 7 wherein the composition formulating oral administration.

13) The pharmaceutical composition of claim 7 wherein the formulation having a liquid, lotion, cream, spray, gel, ointment, or powder composition.

14) The pharmaceutical composition of claim 7 wherein the formulation administering to an animal patient.

15) The pharmaceutical composition of claim 14 wherein the animal patient is mammalian.

16) The pharmaceutical composition of claim 7 wherein the formulation having a topical lotion, cream, soap, or wipe composition.

17) The pharmaceutical composition of claim 7 wherein the formulation delivery method including oral entailing encapsulation of polypeptide or peptide in microspheres or proteinoids, aerosol, or transdermal.

18) The pharmaceutical composition of claim 17 wherein the oral delivery administering capsules, tablets, gels, pastes, or ointments.

19) The pharmaceutical composition of claim 17 wherein the transdermal administering ointments, cream, lotion or patch.

20) The pharmaceutical composition of claim 19 wherein the cream or lotion consisting of at least ten percent salt or mud.
